SEQUENCE LISTING (26 sequences)

SEQ ID NO: 1 - RNA, 21 nt, Artificial Sequence
  artificial construct
  misc_RNA (1)..(21): siRNA
aauuauuaaccacauaagcca

SEQ ID NO: 2 - RNA, 21 nt, Artificial Sequence
  artificial construct
  misc_RNA (1)..(21): siRNA
gcuuaugugguuaauaauuaa

SEQ ID NO: 3 - RNA, 21 nt, Artificial Sequence
  artificial construct
  misc_RNA (1)..(21): siRNA
uaauucuaagcauguuaggca

SEQ ID NO: 4 - RNA, 21 nt, Artificial Sequence
  artificial construct
  misc_RNA (1)..(21): siRNA
ccuaacaugcuuagaauuaug

SEQ ID NO: 5 - RNA, 21 nt, Artificial Sequence
  artificial construct
  misc_RNA (1)..(21): siRNA
ucauaaacggauuauagacgu

SEQ ID NO: 6 - RNA, 21 nt, Artificial Sequence
  artificial construct
  misc_RNA (1)..(21): siRNA
gucuauaauccguuuaugauu

SEQ ID NO: 7 - RNA, 21 nt, Artificial Sequence
  artificial construct
  misc_RNA (1)..(21): siRNA
  misc_feature (1)..(1): 2′-O-Methylnucleoside
  misc_feature (2)..(2): 2′-Fluoronucleoside
  misc_feature (4)..(4): 2′-Fluoronucleoside
  misc_feature (10)..(10): 2′-Fluoronucleoside
  misc_feature (13)..(13): 2′-O-Methylnucleoside
  misc_feature (15)..(15): 2′-O-Methylnucleoside
  misc_feature (16)..(21): Phosphorothioate bond
aauuauuaaccacauaagcca

SEQ ID NO: 8 - DNA, 21 bp, Artificial Sequence
  artificial construct
  misc_RNA (1)..(21): siRNA
  misc_feature (2)..(4): 2′-O-Methylnucleoside
  misc_feature (12)..(12): 2′-O-Methylnucleoside
  misc_feature (18)..(19): 2′-O-Methylnucleoside
  misc_feature (19)..(21): Phosphorothioate bond
gcuuaugugguuaauaauutt

SEQ ID NO: 9 - RNA, 21 nt, Artificial Sequence
  artificial construct
  misc_RNA (1)..(21): siRNA
  misc_feature (1)..(1): 2′-O-Methylnucleoside
  misc_feature (2)..(2): 2′-Fluoronucleoside
  misc_feature (4)..(4): 2′-Fluoronucleoside
  misc_feature (13)..(13): 2′-O-Methylnucleoside
  misc_feature (15)..(15): 2′-O-Methylnucleoside
  misc_feature (16)..(21): Phosphorothioate bond
uaauucuaagcauguuaggca

SEQ ID NO: 10 - DNA, 21 bp, Artificial Sequence
  artificial construct
  misc_RNA (1)..(21): siRNA
  misc_feature (2)..(3): 2′-O-Methylnucleoside
  misc_feature (12)..(12): 2′-O-Methylnucleoside
  misc_feature (17)..(18): 2′-O-Methylnucleoside
  misc_feature (19)..(21): Phosphorothioate bond
  misc_feature (19)..(21): Phosphorothioate bond
ccuaacaugcuuagaauuatt

SEQ ID NO: 11 - RNA, 21 nt, Artificial Sequence
  artificial construct
  misc_RNA (1)..(21): siRNA
  misc_feature (1)..(1): 2′-O-Methylnucleoside
  misc_feature (2)..(2): 2′-Fluoronucleoside
  misc_feature (4)..(4): 2′-Fluoronucleoside
  misc_feature (13)..(13): 2′-O-Methylnucleoside
  misc_feature (15)..(15): 2′-O-Methylnucleoside
  misc_feature (16)..(21): Phosphorothioate bond
ucauaaacggauuauagacgu

SEQ ID NO: 12 - DNA, 21 bp, Artificial Sequence
  artificial construct
  misc_RNA (1)..(21): siRNA
  misc_feature (2)..(4): 2′-O-Methylnucleoside
  misc_feature (13)..(14): 2′-O-Methylnucleoside
  misc_feature (17)..(17): 2′-O-Methylnucleoside
  misc_feature (19)..(21): Phosphorothioate bond
gucuauaauccguuuaugatt

SEQ ID NO: 13 - RNA, 21 nt, Artificial Sequence
  artificial construct
  misc_RNA (1)..(21): siRNA
  misc_feature (1)..(1): 2′-O-Methylnucleoside
  misc_feature (2)..(2): 2′-Fluoronucleoside
  misc_feature (4)..(4): 2′-Fluoronucleoside
  misc_feature (10)..(10): 2′-Fluoronucleoside
  misc_feature (13)..(13): 2′-O-Methylnucleoside
  misc_feature (15)..(15): 2′-O-Methylnucleoside
  misc_feature (16)..(21): Phosphorothioate bond
aauuauuaaccacauaagcca

SEQ ID NO: 14 - DNA, 21 bp, Artificial Sequence
  artificial construct
  misc_RNA (1)..(21): siRNA
  misc_feature (2)..(4): 2′-O-Methylnucleoside
  misc_feature (12)..(12): 2′-O-Methylnucleoside
  misc_feature (18)..(18): 2′-O-Methylnucleoside
  misc_feature (19)..(19): 4′-aminoethyl-2′-fluorouridine
  misc_feature (19)..(21): Phosphorothioate bond
gcuuaugugguuaauaauutt

SEQ ID NO: 15 - DNA, 21 bp, Artificial Sequence
  Artificial construct
  misc_feature (1)..(1): 2′-O-Methylnucleoside
  misc_RNA (1)..(21): siRNA
  misc_feature (2)..(2): 2′-Fluoronucleoside
  misc_feature (4)..(4): 2′-Fluoronucleoside
  misc_feature (13)..(13): 2′-O-Methylnucleoside
  misc_feature (15)..(15): 2′-O-Methylnucleoside
  misc_feature (16)..(21): Phosphorothioate bond
uaauucuaagcauguuaggca

SEQ ID NO: 16 - DNA, 21 bp, Artificial Sequence
  artificial construct
  misc_RNA (1)..(21): siRNA
  misc_feature (2)..(3): 2′-O-Methylnucleoside
  misc_feature (12)..(12): 2′-O-Methylnucleoside
  misc_feature (17)..(17): 2′-O-Methylnucleoside
  misc_feature (18)..(18): 4′-aminoethyl-2′-fluorouridine
  misc_feature (19)..(21): Phosphorothioate bond
ccuaacaugcuuagaauuatt

SEQ ID NO: 17 - RNA, 21 nt, Artificial Sequence
  artificial construct
  misc_RNA (1)..(21): siRNA
  misc_feature (1)..(1): 2′-O-Methylnucleoside
  misc_feature (2)..(2): 2′-Fluoronucleoside
  misc_feature (4)..(4): 2′-Fluoronucleoside
  misc_feature (13)..(13): 2′-O-Methylnucleoside
  misc_feature (15)..(15): 2′-O-Methylnucleoside
  misc_feature (16)..(21): Phosphorothioate bond
ucauaaacggauuauagacgu

SEQ ID NO: 18 - DNA, 21 bp, Artificial Sequence
  artificial construct
  misc_RNA (1)..(21): siRNA
  misc_feature (2)..(4): 2′-O-Methylnucleoside
  misc_feature (13)..(14): 2′-O-Methylnucleoside
  misc_feature (17)..(17): 4′-aminoethyl-2′-fluorouridine
  misc_feature (19)..(21): Phosphorothioate bond
gucuauaauccguuuaugatt

SEQ ID NO: 19 - RNA, 21 nt, Artificial Sequence
  artificial construct
  misc_RNA (1)..(21)
  misc_feature (1)..(1): 2′-O-Methylnucleoside
  misc_feature (2)..(2): 2′-Fluoronucleoside
  misc_feature (4)..(4): 2′-Fluoronucleoside
  misc_feature (10)..(10): 2′-Fluoronucleoside
  misc_feature (13)..(13): 2′-O-Methylnucleoside
  misc_feature (15)..(15): 2′-O-Methylnucleoside
  misc_feature (16)..(21): Phosphorothioate bond
aauuauuaaccacauaagcca

SEQ ID NO: 20 - DNA, 21 bp, Artificial Sequence
  artificial construct
  misc_RNA (1)..(21): siRNA
  misc_feature (1)..(1): coupled to a spermine molecule
  misc_feature (2)..(4): 2′-O-Methylnucleoside
  misc_feature (12)..(12): 2′-O-Methylnucleoside
  misc_feature (18)..(18): 2′-O-Methylnucleoside
  misc_feature (19)..(19): 4′-aminoethyl-2′-fluorouridine
  misc_feature (19)..(21): Phosphorothioate bond
gcuuaugugguuaauaauutt

SEQ ID NO: 21 - RNA, 21 nt, Artificial Sequence
  artificial construct
  misc_RNA (1)..(21): siRNA
  misc_feature (1)..(1): 2′-O-Methylnucleoside
  misc_feature (2)..(2): 2′-Fluoronucleoside
  misc_feature (4)..(4): 2′-Fluoronucleoside
  misc_feature (13)..(13): 2′-O-Methylnucleoside
  misc_feature (15)..(15): 2′-O-Methylnucleoside
  misc_feature (16)..(21): Phosphorothioate bond
uaauucuaagcauguuaggca

SEQ ID NO: 22 - DNA, 21 bp, Artificial Sequence
  artificial construct
  misc_RNA (1)..(21): siRNA
  misc_feature (1)..(1): coupled to a spermine molecule
  misc_feature (2)..(3): 2′-O-Methylnucleoside
  misc_feature (12)..(12): 2′-O-Methylnucleoside
  misc_feature (17)..(17): 2′-O-Methylnucleoside
  misc_feature (18)..(18): 4′-aminoethyl-2′-fluorouridine
  misc_feature (19)..(21): Phosphorothioate bond
ccuaacaugcuuagaauuatt

SEQ ID NO: 23 - RNA, 21 nt, Artificial Sequence
  artificial construct
  misc_RNA (1)..(21): siRNA
  misc_feature (1)..(1): 2′-O-Methylnucleoside
  misc_feature (2)..(2): 2′-Fluoronucleoside
  misc_feature (4)..(4): 2′-Fluoronucleoside
  misc_feature (13)..(13): 2′-O-Methylnucleoside
  misc_feature (15)..(15): 2′-O-Methylnucleoside
  misc_feature (16)..(21): Phosphorothioate bond
ucauaaacggauuauagacgu

SEQ ID NO: 24 - DNA, 21 bp, Artificial Sequence
  artificial construct
  misc_RNA (1)..(21): siRNA
  misc_feature (1)..(1): coupled to a spermine molecule
  misc_feature (2)..(4): 2′-O-Methylnucleoside
  misc_feature (13)..(14): 2′-O-Methylnucleoside
  misc_feature (17)..(17): 4′-aminoethyl-2′-fluorouridine
  misc_feature (19)..(21): Phosphorothioate bond
gucuauaauccguuuaugatt

SEQ ID NO: 25 - RNA, 21 nt, Artificial Sequence
  artificial construct
  misc_RNA (1)..(21): siRNA
  misc_feature (1)..(1): 2′-O-Methylnucleoside
  misc_feature (2)..(2): 2′-Fluoronucleoside
  misc_feature (4)..(4): 2′-Fluoronucleoside
  misc_feature (13)..(13): 2′-O-Methylnucleoside
  misc_feature (15)..(15): 2′-O-Methylnucleoside
  misc_feature (16)..(21): Phosphorothioate bond
ucauaaacggauuauagacgu

SEQ ID NO: 26 - DNA, 21 bp, Artificial Sequence
  artificial construct
  misc_RNA (1)..(21): siRNA
  misc_feature (2)..(4): 2′-O-Methylnucleoside
  misc_feature (13)..(13): 2′-O-Methylnucleoside
  misc_feature (14)..(14): 5′-aminopropyl-2′-OMe-uridine
  misc_feature (17)..(17): 5′-aminopropyl-2′-OMe-uridine
  misc_feature (19)..(21): Phosphorothioate bond
gucuauaauccguuuaugatt

(19) United States
(12) Patent Application Publication
(10) Pub. No.: US 2023/0323355 A1
(43) Pub. Date: Oct. 12, 2023

(54) ANTIVIRAL SILENCING RNA MOLECULES, CHEMICALLY MODIFIED ANTIVIRAL SILENCING RNA MOLECULES WITH ENHANCED CELL PENETRATING ABILITIES, PHARMACEUTICAL COMPOSITIONS COMPRISING SAME AND USES THEREOF FOR TREATMENT OF VIRAL INFECTIONS

(71) Applicant: SOLSTAR PHARMA INC., Laval (CA)

(72) Inventors: Dionissios BALTZIS, Laval (CA); Yasuhiro FURUICHI, Kamakura (JP); Tokuhiro CHANO, Kusatsu, Shiga (JP); Yoshihito UENO, Gifu (JP); Kumiko UI-TEI, Tokyo (JP); Max ARELLA, Laval (CA)

(73) Assignee: SOLSTAR PHARMA INC., Laval, QC (CA)

(21) Appl. No.: 18/024,641

(22) PCT Filed: Sep. 3, 2021
(86) PCT No.: PCT/IB2021/058070

§ 371 Date: Mar. 3, 2023

Related U.S. Application Data
(60) Provisional application No. 63/074,619, filed on Sep. 4, 2020.

(51) Int. Cl. C12N 15/113; A61K 31/713; A61K 47/34; A61K 47/26; A61K 47/18; A61P 31/14; A61K 31/675; A61K 9/00

(52) U.S. Cl. CPC C12N 15/1131; A61K 31/713; A61K 47/34; A61K 47/26; A61K 47/183; A61P 31/14; A61K 31/675; A61K 9/0043; C12N 2310/14; C12N 2310/315; C12N 2310/321; C12N 2310/33; C12N 2310/351

(57) ABSTRACT

Described herein are antiviral silencing RNA molecules (siRNAs), pharmaceutical compositions comprising same and uses thereof for the treatment of viral infections. In embodiments the siRNAs comprises chemically modification(s) for enhanced cell penetrating abilities and/or for greater nuclease resistance. Examples of chemical modifications include substituting one or more nucleotides of a native siRNA molecule with 2′-O-Methylnucleoside, 2′-Fluoronucleoside, aminoalkyl-nucleotide, aminoethyl-nucleotide, and/or 5′-aminopropyl-2′-OMe-nucleoside. The chemically modified nucleotides may be incorporated into the P strand, the G strand or both. Other possible modifications include coupling a compound such as a spermine molecule to the 5′ terminus or the 3′ terminus of the siRNA.

### CROSS REFERENCE TO RELATED APPLICATION

The present application claims priority to United States provisional patent application U.S. 63/074,619 filed on Sep. 4, 2020, which content is incorporated herein in its entirety.

### FIELD OF THE INVENTION

The invention relates to the field of viral infections, and more particularly to antiviral small interfering RNA (siRNA) molecules and uses thereof.

### BACKGROUND OF THE INVENTION

RNA interference (RNAi) is a biological process in which RNA molecules inhibit gene expression or translation by degrading targeted mRNA molecules. Small interfering RNA (siRNA), sometimes known as short interfering RNA or silencing RNA, is a class of double-stranded RNA molecules, typically 20-25 base pairs in length, operating within the RNA interference (RNAi) pathway. The siRNA interferes with the expression of specific genes with complementary nucleotide sequences by degrading mRNA, preventing translation.

Recently a new class of innovative medicines, known as RNAi pharmaceuticals, has emerged. For instance, international PCT publications WO 2018/110678 and WO 2019/088179 describe siRNA molecules that comprise chemically modified nucleotides which help siRNA molecules penetrate mammalian cells and suppress expression of distinct genes for cancer therapy.

The COVID-19 pandemic, also known as the coronavirus pandemic, is an ongoing global pandemic of coronavirus disease 2019 (COVID-19), caused by a single-stranded RNA virus referred to as severe acute respiratory syndrome coronavirus 2 (SARS-CoV-2). Although some candidate compounds are currently being tested for antiviral activity against SARS-CoV-2, no treatment has been identified yet.

There is thus an urgent need for new antiviral agents, and particularly antiviral agents that are effective against coronaviruses such as SARS-CoV-2.

There is also a need for antiviral RNAi pharmaceuticals that can penetrate infected mammalian cells and suppress viral gene expression and/or suppress virus replication in the sequence-specific manner.

There is also a need for new oligonucleotides and chemically modified nucleotide sequences useful as RNA drugs, particularly siRNA molecules with enhanced cell penetrating abilities.

There is also a need for siRNAs that can be incorporated into mammalian cells, particularly the SARS-CoV-2-infected cells, without the presence of a drug delivery system (DDS) reagent such as lipofectamine or liposomes. There is also a need for siRNAs that have been conjugated to spermine for enhanced cell permeability.

The present invention addresses these needs and other needs as it will be apparent from the review of the disclosure and description of the features of the invention hereinafter.

### BRIEF SUMMARY OF THE INVENTION

According to one aspect, the invention relates to antiviral silencing RNA molecules (siRNAs). The antiviral siRNA molecules comprise a nucleotide sequence that targets a virus, they can penetrate infected mammalian cells and they can inhibit and/or silence gene expression and/or translation of viral genes.

Preferably, the siRNA molecules can penetrate mammalian cells without assistance of a drug delivery system (DDS) reagent.

According to another aspect, the invention relates to the use of aminoalkyl-nucleotides and other chemically modified nucleotides or chemically modified nucleosides in the synthesis of siRNA molecules in order to enhance the penetration of the siRNA into the mammalian cells and/or augment nuclease resistance and/or suppress an immune response of the host against the siRNA, e.g. as compared to a siRNA molecule not comprising one or more of such aminoalkyl-nucleotide(s).

According to another aspect, the invention relates to a method for increasing antiviral activity of a native siRNA molecule, the method comprising introducing at least one chemical modification increasing antiviral activity to the native siRNA molecule. For instance, the method may comprise chemically modifying the native siRNA molecule for enhancing penetration of the siRNA molecule into target mammalian cells. The chemical modification may comprise substituting one or more nucleotides of the native siRNA molecule with at least one chemically modified amino-alkyl-nucleotide (AA-Nt) and/or linking a compound (e.g. a spermine molecule) to the 5′ terminus or the 3′ terminus of the siRNA.

According to particular aspects, the invention relates to siRNAs molecules identified herein as siRNAs #301, #302, #303, #304, #305, #306, #307§, #308§, #309§, #307, #308, #309, #313 and #314.

According to further particular aspects, the invention relates to isolated nucleic acid molecules comprising any one of SEQ ID NOs: 1 to 26.

According additional aspects, the invention relates to pharmaceutical compositions comprising an antiviral siRNA and/or an isolated nucleic acid molecules as defined herein, and at least one pharmaceutically acceptable carrier, diluent, vehicle, excipient or dispersion enhancer. In one embodiment the pharmaceutical composition is formulated as a dry powder formulation for administration by inhalation. In one embodiment the pharmaceutical composition comprises siRNAs molecules, polyethyleneimine, an excipient such as D-mannitol and a dispersion enhancer such as L-leucine.

A related aspect concerns the use of an antiviral siRNA or to the use of a pharmaceutical composition as defined herein, in the treatment of a viral disease of a mammalian subject in need thereof.

According additional aspects, the invention relates to nucleotide molecules having a definite sequence, to the uses of such nucleotides molecules as is or in the preparation of antiviral siRNAs, and to the use of a pharmaceutical composition comprising same, as defined herein, in: (i) treating viral infections in subjects; (ii) preventing, interfering and/or reducing viral infections and/or propagation in subjects, (iii) preventing, interfering and/or reducing virus transcription and/or viral genome RNA replication; and/or (iv) reducing the load of viruses infection in a subject.

According to an additional related aspect, the invention relates to a method for treating a viral infection, comprising administering to a subject in need a therapeutically effective amount of one or more antiviral siRNA as defined herein, or one or more isolated nucleic acid molecule as defined, or of a pharmaceutical composition as defined herein.

According to another aspect, the invention relates to use of spermine to augment the incorporation of siRNA into cells. A related aspect concern spermine-siRNA conjugated molecules.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

In order for the invention to be readily understood, embodiments of the invention are illustrated by way of example in the accompanying figures.

FIG. 1 shows the chemical structures of 4′-Aminoalkyl-2′-fluoroUridine (left) and 4′-Aminoalkyl-2′-fluoroCytidine (right).

FIG. 2 is a schematic representation of the chemical structure of Spermine-conjugated-aminoalkyl-uridine-built-in-anti-SARS-CoV-2-siRNAs (SP-AA-COVID-siRNA) identified as siRNAs #307, #308 and #309.

FIG. 3 is a bar graph showing inhibition of SARS-CoV-2 replication, in virus-infected Vero cells, by chemically modified siRNAs identified as #307, #308 and #309 according to one particular embodiment of the invention.

FIGS. 4A and 4B are bar graphs antiviral activity of siRNAS #307, #308 and #309 alone or in combination with Remdesivir (REM) (VEKLURY®), in accordance with Example 3.

FIG. 5 is a curve graph showing virus inhibition of increasing concentration of siRNA #309 as measured by qRT-PCR, in accordance with Example 3.

FIG. 6A is a line graph depicting average weight loss of groups hamsters as a percent of starting weight during testing of in vivo efficacy of siRNA #309, in accordance with Example 4.

FIG. 6B is a bar graph showing live viral particle titers as assessed by viral plaque assay on Vero E6 cells, in plaque forming units (PFU) per gram of lung tissue in hamsters, during testing of in vivo efficacy of siRNA #309, in accordance with Example 4.

FIG. 6C is a bar graph showing average fold reduction of viral loads in lung from hamster given vehicle, low, medium and high doses of siRNA #309, in accordance with Example 4. SARS-CoV2 RNA loads in the lung were unchanged.

FIG. 6D is a scatted dot graph showing viral titers on day 5 post-infection following intranasal treatment with linear therapies animal trial of siRNA #309 antiviral against SARS-CoV-2/COVID-19 in accordance with Example 4. The graph shows viral titer (concentration) in experimental cohorts of Syrian Golden hamsters, measured by plaque assay methodology, in plaque forming units (PFUs) The data is represented logarithmically (5=100,000; 4=10,000; 3=1,000; etc.). The black circles, representing the control group (no treatment), show an average viral titer of 5 logs (100,000 PFUs) in each untreated hamsters lungs. The triangles, representing the animals treated with a “medium” dose of Compound #309 (0.675 mg), show an approximate 80% reduction in viral titer (4.3 logs/20,000 PFUs). The diamonds, representing the “high” dose of Compound #309 (1.35 mg) show an average 99% reduction in viral titer (3 logs/1,000 PFUs). The “low” dose (squares) had no significant effects and was comparable to vehicle control. The two dots along the bottom of the graph represent one animal in each of the “medium” and “high” dosage groups where the viral titer in the treated animal's lungs was ˜0, representing complete viral elimination by the treatment.

FIG. 7 is a schematic representation of the chemical structure of aminoalkyl-uridine-built-in-anti-SARS-CoV-2-siRNAs (AA-COVID-siRNA), without a spermine linker, identified as siRNAs #313 and #314.

### DETAILED DESCRIPTION OF EMBODIMENTS

In the following description of the embodiments, references to the accompanying figures are illustrations of an example by which the invention may be practiced. It will be understood that other embodiments may be made without departing from the scope of the invention disclosed.

### General Overview

The invention pertains to new antiviral agents, and particularly antiviral agents that are effective against coronaviruses such as SARS-CoV-2.

According to one aspect, the invention consists of antiviral siRNAs that can penetrate infected mammalian cells, without the presence of a drug delivery system (DDS) reagent such as lipofectamine, and that can suppress viral gene expression and/or suppress virus replication. These antivirals siRNAs are oligonucleotides molecules that have been chemically modified for enhanced cell penetration ability. These antivirals siRNAs also comprise nucleotide sequences targeting mRNAs and RNA genome of viruses that are essential for virus transcription and/or for viral genome RNA replication.

Antiviral siRNAs

One particular aspect of the invention concerns small RNA oligonucleotide molecules which nucleotide sequence is designed to act as an antiviral siRNA, i.e. a silencing RNA molecule (i.e. siRNA) that can target and degrade viral mRNA molecules and thereby inhibit or silence gene expression or translation of viral genes.

Preferably, the silencing RNA molecule has a sequence that is designed for having a strict complementarity with mRNA sequences and genomic sequence of the virus, while avoiding off-target hybridization to the host genomic and mRNA sequences. Accordingly, because of sequence-specificity, the siRNA molecule of the present invention may help to minimize or avoid adverse effects to the host cells, and/or provide potential combination therapies.

Technically, all the mammalian viruses that are known to infect mammalian cells are the target of siRNA therapy in accordance with the present invention. All mammalian viruses, as well as plant viruses can produce mRNAs that are essential for protein synthesis and their growth in the infected cells. As long as the nucleotide sequence of the siRNA matches with essential and indispensable viral mRNAs, such virus will lose the important mRNA and never be able to replicate in the infected cell. Therefore a siRNA molecule according to the invention may target all categories of mammalian and plant viruses, including DNA (double- or single-stranded) and RNA (double- or single-stranded) or single-stranded RNA (with plus polarity or minus polarity to mRNA), as long as the nucleotide sequence of the siRNA is appropriately selected.

According to one particular embodiment, the silencing RNA molecule is directed against a corona virus. In embodiments, the silencing RNA molecule is directed against corona viruses, including lethal varieties that can cause SARS (i.e. SARS-CoV or SARS-CoV-1), MERS (i.e. MERS-CoV), and COVID-19 (i.e. SARS-CoV-2). In embodiments, the silencing RNA molecule enables a complete inhibition of virus replication of one or more of these viruses. In embodiments, the siRNA molecule is effective (i.e. it cross-reacts) against more than one corona virus.

In a preferred embodiment, the virus is SARS-CoV-2 and the silencing RNA molecule has a strict complementarity with sequence(s) of that virus. The entire genomic sequence of SARS-CoV-2 contains about 30,000 nucleotides (GenBank accession number NC_045512). Preferred targeted regions are regions of the SARS-CoV-2 RNA genome that encode critical regions of virus, such as RNA-dependent-RNA polymerase (RdRp) regions essential for the virus transcription, as well as the genome RNA replication. In one particular embodiment, the target site of the siRNA molecule is ORF1ab coding nsp14 3′-5′Exo exonuclease and mRNA capping enzyme NMT. In another particular embodiment, the target site of the siRNA molecule is ORF1 b coding RdRP nsp3. In another particular embodiment, the target site of the siRNA molecule is ORF1b coding RdRP nsp12, NiRAN before Finger. Sequences for these target sites and other sites can be obtained by consulting the entire genomic sequence of SARS-CoV-2 (GenBank accession number NC_045512).

In particular embodiments defined herein, siRNAs #301, #304 and #307 targets with similarly nucleotide sequences to ADRP, nsp3 coded in ORF1ab of SARS-CoV2 genome (NC_045512). In particular embodiments defined herein, siRNAs #302, #305, and #308 target NiRAN and RdRP, nsp12 coded in ORF1ab. In particular embodiments defined herein, siRNAs #303, #306, #309 target 3′-5′ exonuclease, nsp 14 in ORF1ab.

In embodiments, the siRNA molecule of the invention is a double-stranded RNA molecule. In embodiments, the siRNA molecule comprises 19, 20, 21, 22, 23, 24, 25, 26 or 27 base pairs. In embodiments, the siRNA molecule of the invention further comprises 2, 3, 4, or 5 TT nucleotides overhangs at the 5′- or at the 3′-end of the molecule. In preferred embodiments, the siRNA molecule comprises a TT overhang at the 3′-terminus of the molecule. In particular embodiments, the siRNA molecule comprises 19 base pairs plus a TT overhang at the 3′ terminus. In one particular embodiment, the siRNA molecule comprises 19 base pairs plus two TT at each of the 3′-terminus of the molecule, for a total of 42 nucleotides.

In embodiments, the siRNA molecule has a sequence that is designed for having a complete complementarity with a mRNA sequence of SARS-CoV-2 and it is capable of interfering with the expression of specific genes of that virus. In particular embodiments, the siRNA molecule of the present invention is a double-stranded RNAs structure which comprises one of the following sequence:

| | SEQ | |
|---|---|---|
| SIRNA | ID | |
| # | NO: | SEQUENCES |
| 301 | 1 | G: 5′-AAU UAU UAA CCA CAU AAG CCA-3′ |
| | 2 | P: 3′-AA UUA AUA AUU GGU GUA UUC G-5′ |
| 302 | 3 | G: 5′-UAA UUC UAA GCA UGU UAG GCA-3′ |
| | 4 | P: 3′-GU AUU AAG AUU CGU ACA AUC C-5′ |
| 303 | 5 | G: 5′-UCA UAA ACG GAU UAU AGA CGU-3′ |
| | 6 | P: 3′-UU AGU AUU UGC CUA AUA UCU G-5′ |

In embodiments, the siRNA molecule has a sequence that is designed for having complementarity with a mRNA sequence of SARS-CoV-2, and it is also capable of interfering with the expression of specific genes of any one or both of SARS-CoV-1 and/or MERS-CoV. For instance, sequences #303, #306, #309, #313 and #314 are siRNA sequences having some homology to SARS-CoV-1 and/or MERS-CoV and it may well inhibit the growth of both of these viruses.

In embodiments, the siRNA molecule of the present invention comprises a nucleotide sequence that has been chemically modified, for instance to comprise chemically modified nucleotides and or to comprise a molecule or compound coupled to the 5′ terminus or the 3′ terminus of the to the P strand and/or the G strand of the siRNA.

Advantageous modifications to the siRNA sequence may include, but are not limited to, modifications that confer one or more of ribonuclease resistance, increased stability, increased cellular permeability to mammalian cells, reduce and/or suppress an host immune response against the siRNA, etc. (e.g. as compared to a siRNA molecule not comprising one or more of such modifications).

In embodiments, the siRNA sequence includes one or more 2′-O-methylation, a common nucleoside modification of RNA where a methyl group is added to the 2′ hydroxyl of the ribose moiety of a nucleoside, producing a methoxy group. Such modification and the like may be useful, for instance to increase nuclease resistance and/or stabilize the aminoalkyl nucleotide.

In embodiments, the siRNA sequence includes one or more 2′-fluorinatednucleoside, a common nucleoside modification of RNA where a fluoro group is added to the 2′ hydroxyl of the ribose moiety of a nucleoside. Such modification and the like may be useful, for instance to increase nuclease resistance and/or stabilize the aminoalkyl nucleotide and/or reduce and/or suppress a host immune response against the siRNA.

In embodiments, the siRNA sequence include one or more 4′-aminoethyl-2′-fluoronucleotide, including but not limited to 4′-aminoethyl-2′-fluorouridine (e.g. see FIG. 2 and FIG. 7). Such modification and the like may be useful, for instance to increase nuclease resistance and/or increase cellular permeability to mammalian cells.

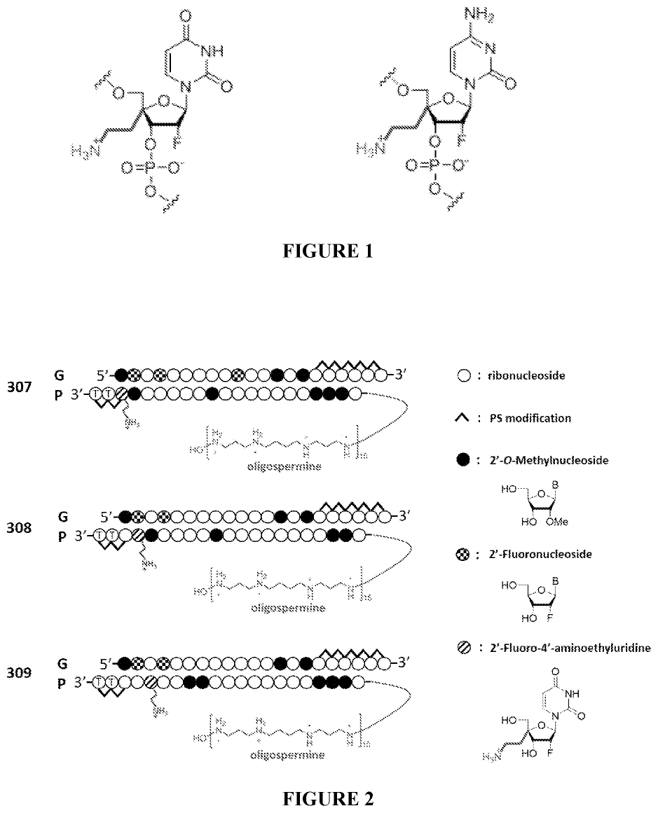

In one particular embodiment, the siRNA molecule comprises one or more chemically modified nucleotides that increases cellular permeability and/or enhance cell penetration abilities of the siRNA molecule into mammalian cells. Examples include, but are not limited to, chemically modified amino-alkyl-nucleotides (AA-Nt) such as 4′-Aminoalkyl-2′-fluoroUridine and 4′-Aminoalkyl-2′-fluoroCytidine (FIG. 1). Manufacture and incorporation of these two nucleotides into siRNAs have been described in detail in international PCT publications WO 2018/110678 and WO 2019/088179, as well as in Japanese Patent application No. 2019-148061 (filed on Aug.8^{th} 2019), these patent applications being incorporated herein by reference. The chemically modified nucleotides may be incorporated into the P strand (i.e. passenger stand or plus sense), the G strand (i.e. guide strand or anti-sense) or both.

In one particular embodiment, the siRNA molecule comprises one or more chemically modified nucleotides that increases stability of the siRNA, for instance thermal stability and/or resistance to nucleases degradation. In embodiments, the siRNAs are modified to comprise at least one 5′-aminopropyl-2′-OMe-nucleoside, including but not limited to a 5′-aminopropyl-2′-OMe-uridine (FIG. 7). Manufacture and incorporation of this chemically modified nucleoside into siRNAs have been described in detail in PCT patent publication WO 2019/088179, and in Kajino et al., J. Org. Chem. 2019 Mar. 15; 84(6):3388-3404 (doi: 10.1021/acs.joc.8b03277). The chemically modified nucleotides may be incorporated into the P strand (i.e. passenger stand or plus sense), the G strand (i.e. guide strand or anti-sense) or both.

The number and the positioning of the one or more chemically modified nucleotides in the siRNA molecule can be optimised by those skilled in the art, for instance using the instructions and methodology described by Koizumi et al. (2018), Synthesis of 4′-aminoalkyl-2′-O-methyl modified RNA and their biological properties, Bioorganic & Medicinal Chemistry 26,3521-3534; and/or by Kano et al. (2018), Synthesis and properties of 4′-C-aminoalkyl-2′-fluoro-modified RNA oligomers, Bioorganic & Medicinal Chemistry 26,4574-4582, both incorporated herein by reference. Reasons for optimization may include, but are not limited to, increasing nuclease resistance of the siRNA, increasing gene expression suppression ability, and/or improving cell membrane permeability. In embodiments, the siRNA is a 21-mer that comprises one or more chemical modified nucleotide(s) that is(are) positioned within the siRNA molecule in accordance to Table 1, wherein a bullet ( ) represents a modified chemical nucleotide:

TABLE 1
Example of positioning of chemical modified nucleotide within a 21-mer siRNA

| | 1 | 2 | 3 | 4 | 5 | 6 | 7 | 8 | 9 | 10 | 11 | 12 | 13 | 14 | 15 | 16 | 17 | 18 | 19 | 20 | 21 | |
|---|---|---|---|---|---|---|---|---|---|---|---|---|---|---|---|---|---|---|---|---|---|---|
| (5′)- | • | X | X | X | X | X | X | X | X | X | X | X | X | X | X | X | X | X | X | T | T | -(3′) |
| (5′)- | X | • | X | X | X | X | X | X | X | X | X | X | X | X | X | X | X | X | X | T | T | -(3′) |
| (5′)- | X | X | • | X | X | X | X | X | X | X | X | X | X | X | X | X | X | X | X | T | T | -(3′) |
| (5′)- | X | X | X | • | X | X | X | X | X | X | X | X | X | X | X | X | X | X | X | T | T | -(3′) |
| (5′)- | X | X | X | X | • | X | X | X | X | X | X | X | X | X | X | X | X | X | X | T | T | -(3′) |
| (5′)- | X | X | X | X | X | • | X | X | X | X | X | X | X | X | X | X | X | X | X | T | T | -(3′) |
| (5′)- | X | X | X | X | X | X | • | X | X | X | X | X | X | X | X | X | X | X | X | T | T | -(3′) |
| (5′)- | X | X | X | X | X | X | X | • | X | X | X | X | X | X | X | X | X | X | X | T | T | -(3′) |
| (5′)- | X | X | X | X | X | X | X | X | • | X | X | X | X | X | X | X | X | X | X | T | T | -(3′) |
| (5′)- | X | X | X | X | X | X | X | X | X | • | X | X | X | X | X | X | X | X | X | T | T | -(3′) |
| (5′)- | X | X | X | X | X | X | X | X | X | X | • | X | X | X | X | X | X | X | X | T | T | -(3′) |
| (5′)- | X | X | X | X | X | X | X | X | X | X | X | • | X | X | X | X | X | X | X | T | T | -(3′) |
| (5′)- | X | X | X | X | X | X | X | X | X | X | X | X | • | X | X | X | X | X | X | T | T | -(3′) |
| (5′)- | X | X | X | X | X | X | X | X | X | X | X | X | X | • | X | X | X | X | X | T | T | -(3′) |
| (5′)- | X | X | X | X | X | X | X | X | X | X | X | X | X | X | • | X | X | X | X | T | T | -(3′) |
| (5′)- | X | X | X | X | X | X | X | X | X | X | X | X | X | X | X | • | X | X | X | T | T | -(3′) |
| (5′)- | X | X | X | X | X | X | X | X | X | X | X | X | X | X | X | X | • | X | X | T | T | -(3′) |
| (5′)- | X | X | X | X | X | X | X | X | X | X | X | X | X | X | X | X | X | • | X | T | T | -(3′) |
| (5′)- | X | X | X | X | X | X | X | X | X | X | X | X | X | X | X | X | X | X | • | T | T | -(3′) |
| (5′)- | • | • | X | X | X | X | X | X | X | X | X | X | X | X | X | X | X | X | X | T | T | -(3′) |
| (5′)- | X | • | • | X | X | X | X | X | X | X | X | X | X | X | X | X | X | X | X | T | T | -(3′) |
| (5′)- | X | X | • | • | X | X | X | X | X | X | X | X | X | X | X | X | X | X | X | T | T | -(3′) |
| (5′)- | X | X | X | • | • | X | X | X | X | X | X | X | X | X | X | X | X | X | X | T | T | -(3′) |
| (5′)- | X | X | X | X | • | • | X | X | X | X | X | X | X | X | X | X | X | X | X | T | T | -(3′) |
| (5′)- | X | X | X | X | X | • | • | X | X | X | X | X | X | X | X | X | X | X | X | T | T | -(3′) |
| (5′)- | X | X | X | X | X | X | • | • | X | X | X | X | X | X | X | X | X | X | X | T | T | -(3′) |
| (5′)- | X | X | X | X | X | X | X | • | • | X | X | X | X | X | X | X | X | X | X | T | T | -(3′) |
| (5′)- | X | X | X | X | X | X | X | X | • | • | X | X | X | X | X | X | X | X | X | T | T | -(3′) |
| (5′)- | X | X | X | X | X | X | X | X | X | • | • | X | X | X | X | X | X | X | X | T | T | -(3′) |
| (5′)- | X | X | X | X | X | X | X | X | X | X | • | • | X | X | X | X | X | X | X | T | T | -(3′) |
| (5′)- | X | X | X | X | X | X | X | X | X | X | X | • | • | X | X | X | X | X | X | T | T | -(3′) |
| (5′)- | X | X | X | X | X | X | X | X | X | X | X | X | • | • | X | X | X | X | X | T | T | -(3′) |
| (5′)- | X | X | X | X | X | X | X | X | X | X | X | X | X | • | • | X | X | X | X | T | T | -(3′) |
| (5′)- | X | X | X | X | X | X | X | X | X | X | X | X | X | X | • | • | X | X | X | T | T | -(3′) |
| (5′)- | X | X | X | X | X | X | X | X | X | X | X | X | X | X | X | • | • | X | X | T | T | -(3′) |
| (5′)- | X | X | X | X | X | X | X | X | X | X | X | X | X | X | X | X | • | • | X | T | T | -(3′) |
| (5′)- | X | X | X | X | X | X | X | X | X | X | X | X | X | X | X | X | X | • | • | T | T | -(3′) |
| (5′)- | X | • | • | X | X | X | X | X | X | X | X | X | X | X | X | X | X | X | X | X | X | -(3′) |
| (5′)- | X | • | X | • | X | X | X | X | X | X | X | X | X | X | X | X | X | X | X | X | X | -(3′) |
| (5′)- | X | • | X | X | • | X | X | X | X | X | X | X | X | X | X | X | X | X | X | X | X | -(3′) |
| (5′)- | X | • | X | X | X | • | X | X | X | X | X | X | X | X | X | X | X | X | X | X | X | -(3′) |
| (5′)- | X | • | X | X | X | X | • | X | X | X | X | X | X | X | X | X | X | X | X | X | X | -(3′) |
| (5′)- | X | • | X | X | X | X | X | • | X | X | X | X | X | X | X | X | X | X | X | X | X | -(3′) |
| (5′)- | X | • | X | X | X | X | X | X | • | X | X | X | X | X | X | X | X | X | X | X | X | -(3′) |
| (5′)- | X | • | X | X | X | X | X | X | X | • | X | X | X | X | X | X | X | X | X | X | X | -(3′) |
| (5′)- | X | • | X | X | X | X | X | X | X | X | • | X | X | X | X | X | X | X | X | X | X | -(3′) |
| (5′)- | X | • | X | X | X | X | X | X | X | X | X | X | • | X | X | X | X | X | X | X | X | -(3′) |
| (5′)- | X | • | X | X | X | X | X | X | X | X | X | X | X | • | X | X | X | X | X | X | X | -(3′) |
| (5′)- | X | • | X | X | X | X | X | X | X | X | X | X | X | X | • | X | X | X | X | X | X | -(3′) |
| (5′)- | X | • | X | X | X | X | X | X | X | X | X | X | X | X | X | • | X | X | X | X | X | -(3′) |
| (5′)- | X | • | X | X | X | X | X | X | X | X | X | X | X | X | X | X | • | X | X | X | X | -(3′) |
| (5′)- | X | • | X | X | X | X | X | X | X | X | X | X | X | X | X | X | X | • | X | X | X | -(3′) |
| (5′)- | X | • | X | X | X | X | X | X | X | X | X | X | X | X | X | X | X | X | • | X | X | -(3′) |
| (5′)- | X | • | X | X | X | X | X | X | X | X | X | X | X | X | X | X | X | X | X | • | X | -(3′) |
| (5′)- | X | • | X | X | X | X | X | X | X | X | X | X | X | X | X | X | X | X | X | X | • | -(3′) |
| (5′)- | X | • | X | • | • | X | X | X | X | X | X | X | X | X | X | X | X | X | X | X | X | -(3′) |
| (5′)- | X | • | X | • | X | • | X | X | X | X | X | X | X | X | X | X | X | X | X | X | X | -(3′) |
| (5′)- | X | • | X | • | X | X | • | X | X | X | X | X | X | X | X | X | X | X | X | X | X | -(3′) |
| (5′)- | X | • | X | • | X | X | X | • | X | X | X | X | X | X | X | X | X | X | X | X | X | -(3′) |
| (5′)- | X | • | X | • | X | X | X | X | • | X | X | X | X | X | X | X | X | X | X | X | X | -(3′) |
| (5′)- | X | • | X | • | X | X | X | X | X | • | X | X | X | X | X | X | X | X | X | X | X | -(3′) |
| (5′)- | X | • | X | • | X | X | X | X | X | X | • | X | X | X | X | X | X | X | X | X | X | -(3′) |
| (5′)- | X | • | X | • | X | X | X | X | X | X | X | • | X | X | X | X | X | X | X | X | X | -(3′) |
| (5′)- | X | • | X | • | X | X | X | X | X | X | X | X | • | X | X | X | X | X | X | X | X | -(3′) |
| (5′)- | X | • | X | • | X | X | X | X | X | X | X | X | X | • | X | X | X | X | X | X | X | -(3′) |
| (5′)- | X | • | X | • | X | X | X | X | X | X | X | X | X | X | • | X | X | X | X | X | X | -(3′) |
| (5′)- | X | • | X | • | X | X | X | X | X | X | X | X | X | X | X | • | X | X | X | X | X | -(3′) |
| (5′)- | X | • | X | • | X | X | X | X | X | X | X | X | X | X | X | X | • | X | X | X | X | -(3′) |
| (5′)- | X | • | X | • | X | X | X | X | X | X | X | X | X | X | X | X | X | • | X | X | X | -(3′) |
| (5′)- | X | • | X | • | X | X | X | X | X | X | X | X | X | X | X | X | X | X | • | X | X | -(3′) |
| (5′)- | X | • | X | • | X | X | X | X | X | X | X | X | X | X | X | X | X | X | X | • | X | -(3′) |
| (5′)- | X | • | X | • | X | X | X | X | X | X | X | X | X | X | X | X | X | X | X | X | • | -(3′) |

In embodiments, the siRNA is a 21-mer designed for efficacy against SARS-CoV-2 and it comprises one or more chemically chemical nucleotide(s), e.g. at least one of chemically modified uridine, chemically modified cytidine, chemically modified adenine, chemically modified guanine. According to particular embodiments, the siRNA molecules of the present invention are selected from siRNAs #304, #305 and #306 as follows:

| SIRNA | SEQ ID | |
|---|---|---|
| # | NO: | SEQUENCES* |
| 304 | 7 | G: 5′-aAU UAU UAA CCA cAu A•A•G•C•C•A-3′ |
| | 8 | P: 3′-T•T•uuA AUA AuU GGU GUA uuc G-5′ |
| 305 | 9 | G: 5′-uAA UUC UAA GCA uGu U•A•G•G•C•A-3′ |
| | 10 | P: 3′-T•T•Auu AAG AuU CGU ACA Auc C-5′ |
| 306 | 11 | G: 5′-uCA UAA ACG GAU uAu A•G•A•C•G•U-3′ |
| | 12 | P: 3′-T•T•AGu AUu uGC CUA AUA ucu G-5′ |

*lowercase: 2′-O-Methylnucleoside
Bold: 2′-Fluoronucleoside
dot (•): Phosphorothioate (PS) bond

In embodiments, the siRNAs are modified to comprise at least one of a 4′-aminoethyl-2′-fluorouridine and a 4′-Aminoalkyl-2′-fluorocytidine, which chemical structures are shown in FIG. 1. In accordance with such embodiments, examples of siRNA molecules comprising a 4′-aminoethyl-2′-fluorouridine are identified herein as siRNAs #307§, #308§ and #309§ (#313) as follows:

| SIRNA | SEQ | |
|---|---|---|
| # | ID NO: | SEQUENCES* |
| 307§ | 13 | G: 5′-aAU UAU UAA CCA cAu A•A•G•C•C•A-3′ |
| | 14 | P: 3′-T•T•uuA AUA AuU GGU GUA uuc G-5′ |
| 308§ | 15 | G: 5′-uAA UUC UAA GCA uGu U•A•G•G•C•A-3′ |
| | 16 | P: 3′-T•T•Auu AAG AuU CGU ACA Auc C-5′ |
| 309§ | 17 | G: 5′-uCA UAA ACG GAU uAu A• G•A•C•G•U-3′ |
| (313) | 18 | P: 3′-T•T•AGu AUu uGC CUA AUA ucu G-5′ |

*lowercase: 2′-O-Methylnucleoside
Bold: 2′-Fluoronucleoside
Underlined uridine: 4′-aminoethyl-2′-fluorouridine
dot (•): Phosphorothioate (PS) bond

In embodiments, the siRNAs are modified to comprise at least one 5′-aminopropyl-2′-OMe-nucleoside, including but not limited to a 5′-aminopropyl-2′-OMe-uridine which chemical structure is shown in FIG. 7. One particular example of a siRNA molecule comprising a 5′-aminopropyl-2′-OMe-uridine is identified herein as siRNAs #314 as follows:

| SIRNA | SEQ | |
|---|---|---|
| # | ID NO: | SEQUENCE* |
| 314 | 25 | G: 5′-uCA UAA ACG GAU uAu A•G•A•C•G•U-3′ |
| | 26 | P: 3′-T•T•AG [symbol] AU [symbol] uGC CUA AUA ucu G-5′ |

*lowercase: 2′-O-Methylnucleoside
Bold: 2′-Fluoronucleoside
SMALL CAP ITALIC: 5′-aminopropyl-2′-OMe-uridine
dot (•): Phosphorothioate (PS) bond

Advantageously, siRNAs #307§, #308§ and #309§ (#313) and #314 may display improved nuclease resistance and/or improved cell membrane permeability, as compared to siRNAs #304, #305 and #306 and/or as compared to a native non-modified siRNA.

### Addition of a Spermine Molecule

Spermine is a polyazaalkane that is tetradecane in which the carbons at positions 1, 5, 10 and 14 are replaced by nitrogens. It is found in a wide variety of organisms and tissues and it is an essential growth factor in some bacteria. It is found as a polycation at all pH values. Spermine is associated with nucleic acids, particularly in viruses, and is thought to stabilize the helical structure. Its chemical formula is C₁₀H₂₆N₄.

According to embodiments, a spermine molecule is linked to an antiviral siRNA molecule. Because the resulting spermine-containing siRNA will comprise more positive charges, this spermine-containing siRNA is expected to possess enhanced capabilities for penetration into target mammalian cells and, accordingly, expected to possess an increased antiviral activity.

According to embodiments, a spermine molecule is linked to a siRNA molecule of the invention, including chemically modified siRNAs and specific nucleotide sequences as defined herein. It is also conceivable that spermine may be linked to other siRNAs molecules to provide desired enhanced capabilities for penetration into target mammalian cells and associated antiviral activity.

The spermine molecule may be coupled to the 5′ terminus or the 3′ terminus of the siRNA and it may be coupled to the P strand or the G strand of the siRNA. Methods for coupling a spermine molecule to a siRNA molecule are described hereinafter in the example section, as well as in international patent application PCT/JP2019/148061, which is incorporated herein by reference.

In one particular embodiment, the siRNA is directed against SARS-CoV-2, it comprises the chemically modified sequences siRNAs #307§, #308§ and #309§ as defined above and a spermine molecule is coupled to the 3′ terminus of P strand in these siRNAs. In accordance with such embodiments, the siRNA molecules of the present invention are selected from siRNAs #307, #308 and #309 as follows:

| SIRNA | SEQ | |
|---|---|---|
| # | ID NO: | SEQUENCES* |
| 307 | 19 | G: 5′-aAU UAU UAA CCA cAu A•A•G•C•C•A-3′ |
| | 20 | P: 3′-T•T•uuA AUA AuU GGU GUA uuc G(S₁₅)-5′ |
| 308 | 21 | G: 5′-uAA UUC UAA GCA uGu U•A•G•G•C•A-3′ |
| | 22 | P: 3′-T•T•Auu AAG AuU CGU ACA Auc C(S₁₅)-5′ |
| 309 | 23 | G: 5′-uCA UAA ACG GAU uAu A•G•A•C•G•U-3′ |
| | 24 | P: 3′-T•T•AGu AUu uGC CUA AUA ucu G(S₁₅) -5′ |

*lowercase: 2′-O-Methylnucleoside
Bold: 2′-Fluoronucleoside
Underlined uridine: 4′-aminoethyl-2′-fluorouridine
dot (•): Phosphorothioate (PS) bond
S₁₅: spermine molecule

FIG. 2 illustrates schematically the chemical structure of siRNAs #307, #308 and #309 as defined hereinabove.

The siRNAs molecules of the present invention may also comprise any other molecule that could enhance penetration into target mammalian cells and/or enhance antiviral activity including, but not limited to a tag, a cholesterol molecule, a folic acid molecule, a peptide, etc. linked at the 5′ or 3′ terminus, or both.

Methods for Increasing Antiviral Activity of Native siRNA Molecules

One additional aspect of the invention concerns methods for increasing antiviral activity of siRNA molecules. In embodiments, the increased antiviral activity comprises enhancing penetration of the antiviral siRNA molecule into target mammalian cells.

According to one particular embodiment, the method for increasing antiviral activity of a native siRNA molecule comprises: i) designing a native siRNA molecule with a sequence having complementarity with a virus mRNA; ii) synthesizing a chemically modified antiviral siRNA molecule, the chemically modified antiviral siRNA molecule comprising at least one chemical modification (e.g. a chemical modification that increases antiviral activity and/or a chemical modification that augments nuclease resistance and/or a chemical modification that reduces and/or suppresses an immune response of the host against the siRNA, compared to the native siRNA molecule.

The method may also comprise the additional step of iii) linking at the 5′ terminus of the siRNA or the 3′ terminus, or both, a molecule that can enhance penetration into target mammalian cells and/or enhance antiviral activity. According to embodiments, a spermine molecule is coupled to the 5′ terminus or the 3′ terminus of the siRNA (either the P strand, the G strand, or both of the siRNA). In one particular embodiment, a spermine molecule is coupled to the 5′ terminus of the P strand.

According to preferred embodiments, the designing of the native siRNA molecule with a sequence having complementarity with a virus mRNA comprises identifying specific nucleotide sequences of the siRNA molecules containing distinct sequences hybridisable to the virus (e.g. SARS-CoV-2 virus mRNA) and yet not hybridisable to any of mRNA of virus-infected cells (e.g. human cells). Such sequence(s) may be identified by those skilled in the art using available tools, including for instance the guidelines described previously by one of the inventors, namely Ui-Tei K (Ui-Tei K, Naito Y, Takahashi F, Haraguchi T, Ohki-Hamazaki H, Juni A, Ueda R, Saigo K. (2004) Guidelines for the selection of highly effective siRNA sequences for mammalian and chick RNA interference. Nucleic Acids Res. 32, 936-948).

According to one embodiment, chemically modified antiviral siRNA molecule comprises one or more nucleoside modification including, but not limited to a 2′-O-methylation, a 2′-Fluoro (F) modification, a 4′-Aminoalkyl-2′-fluoro modification and/or a 5′-aminopropyl-2′-Ome modification.

In one embodiment, the at least one chemical modification comprises substituting one or more nucleotides of the native nucleotide in the siRNA molecule with at least one chemically modified amino-alkyl-nucleotide (AA-Nt). The substitution can be made on the P strand, the G strand or both. In embodiments, the amino-alkyl-nucleotides is selected from Aminoalkyl-Uridine, Aminoalkyl-Cytidine, and combination(s) thereof. In preferred embodiments, it is the G strand which is modified.

In one embodiment, the at least one chemical modification comprises substituting one or more nucleotides of the native nucleotide in the siRNA molecule with at least one chemically modified amino-ethyl-fluorinated-nucleotide. The substitution can be made on the P strand, the G strand or both. In embodiments, the amino-ethyl-fluorinated-nucleotide is 4′-aminoethyl-2′-fluorouridine or 4′-Aminoalkyl-2′-fluorocytidine. In preferred embodiments, it is the P strand which is modified with at least one 4′-aminoethyl-2′-fluorouridine.

In one embodiment, the at least one chemical modification comprises substituting one or more nucleotides of the native nucleotide in the siRNA molecule with at least one chemically modified 5′-aminopropyl-2′-Ome-nucleotide. The substitution can be made on the P strand, the G strand or both. In embodiments, the 5′-aminopropyl-2′-Ome-nucleotide is a 5′-aminopropyl-2′-OMe-uridine. In preferred embodiments, it is the P strand which is modified with two 5′-aminopropyl-2′-OMe-uridine.

In another embodiment, the at least one chemical modification comprises coupling a spermine molecule to the 5′ terminus or to the 3′ terminus of the native siRNA or of a modified siRNA already having chemically modified nucleotides. In one preferred embodiment, the at least one chemical modification comprises coupling a spermine molecule to the 5′ terminus of P strand in the siRNA.

If step ii) does not already comprise coupling a molecule to the 5′ terminus or the 3′ terminus of the siRNA, then the method may further comprises the optional step iii) of linking at the 5′ or 3′ terminus, or both of the siRNA a spermine molecule, a tag, a cholesterol molecule, a folic acid molecule, a peptide, or any other molecule that can enhance penetration into target mammalian cells and/or enhance antiviral activity.

Additional modifications that may be envisioned to the siRNAs according to the invention includes increasing nuclease resistance of the siRNA, increasing gene expression suppression ability, and/or improving event further cell membrane permeability.

### Therapeutic Methods and Compositions

Antiviral siRNA molecules in accordance with the present invention may provide substantial therapeutic benefits to subjects, particularly human subjects suffering, or susceptible to suffer, from a viral disease such as COVID-19. Therefore, delivering an antiviral siRNA in accordance with the present invention may have useful pharmaceutical applications in the treatment of viral infections and diseases in mammalian subjects.

Accordingly one additional aspect of the invention concerns the use of an antiviral siRNA as defined herein, in the treatment of a viral disease of a mammalian subject in need thereof. The antiviral siRNAs of the invention may be useful in: (i) treating viral infections (e.g. coronavirus-related disease such as COVID-19) in subjects; (ii) preventing, interfering and/or reducing viral infections and/or propagation in subjects (e.g. a human subject), (iii) preventing, interfering and/or reducing virus transcription and/or viral genome RNA replication; and (iv) reducing the load of viruses infection (e.g. corona viruses) in a subject, particularly in a human subject know (or suspected) of being infected with the virus SARS-CoV-2 causing COVID-19. According to one particular aspect, treatment of a subject comprises reducing viruses replication, thereby reducing the viral load in an infected subject and/or preventing spreading of virus infection to uninfected neighboring cells.

An additional related aspect relates to therapeutic methods (e.g. for treating a viral infection) comprising administering a therapeutically effective amount of one or more antiviral siRNA as described herein. The invention also encompasses methods, compounds, and pharmaceutical compositions for the treatment of a virus infection in a mammal including, but not limited to, corona viruses-related diseases and infections. In embodiments, the viral disease is a corona virus-related disease such as COVID-19.

As used herein, the term “mammalian subject” or “mammalian cell” includes mammals and cells in which treatment against virus infection is desirable. The term “subject” includes domestic animals (e.g. cats, dogs, horses, pigs, cows, goats, sheeps), rodents (e.g. mice or rats), rabbits, squirrels, bears, primates (e.g., chimpanzees, monkeys, gorillas, and humans), wild animals such as those living in zoos (e.g. lion, tiger, elephant, and the like), and transgenic species thereof. Preferably, the mammalian subject is a human, more preferably a human patient in need of treatment. Even more preferably the mammalian subject is a human patient diagnosed or susceptible to suffer from a coronavirus disease such as COVID-1 9, SARS and/or MERS.

As used herein, the terms “treatment” or “treating” of a subject include one or more of administration of an antiviral siRNA to a subject with the purpose of stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being. In one embodiment, the treatment method comprises administering to the subject a therapeutically effective amount of an antiviral siRNA as defined herein.

Related aspects of the invention concern pharmaceutical compositions comprising an effective amount of an antiviral siRNA as defined herein. As used herein, the term “pharmaceutical composition” refers to the presence of at least one of antiviral siRNA molecule as defined herein, and at least one pharmaceutically acceptable carrier, diluent, vehicle or excipient.

One particular aspect concerns the use of a therapeutically effective amount of one or more antiviral siRNA as defined herein for the prevention and/or treatment of a viral disease in mammalian subjects.

As used herein, the term “therapeutically effective amount” or “effective amount” means the amount of compound that, when administered to a subject for treating or preventing a particular disorder, disease or condition, is sufficient to effect such treatment or prevention of that disorder, disease or condition. Dosages and therapeutically effective amounts may vary, for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g. bioavailability, stability, potency, toxicity, etc.), and the particular disorder(s), disease(s) or condition(s) the subject is suffering from. In addition, the therapeutically effective amount may depend on the severity of the disease state, or underlying disease or complications. Such appropriate doses may be determined using any available assays.

When one or more of the antiviral siRNA of the invention is to be administered to humans, a physician may for example, prescribe a relatively low dose at first, subsequently increasing the dose until an appropriate response is obtained.

In embodiments, the effective amount for a human subject may be a single administration of a high concentration of the siRNA(s). Alternatively, the antiviral siRNA may be administered more frequently (e.g. daily, weekly, monthly) and/or until the patient is tested negative for the infection and even afterwards.

“Pharmaceutically acceptable vehicle” refers to a diluent, adjuvant, excipient, carrier or drug delivery substance(s) with which a compound is administered. The term “pharmaceutically acceptable” refers to drugs, medicaments, inert ingredients, etc., which are suitable for use in treatment of a viral infection in mammals (preferably humans) without undue toxicity, incompatibility, instability, irritation, allergic response, and the like, commensurate with a reasonable benefit/risk ratio. It preferably refers to a compound or composition that is approved or approvable by a regulatory agency of the Federal or state government or listed in the U.S. Pharmacopoeia or other generally recognized pharmacopoeia for use in animals and more particularly in humans. The pharmaceutically acceptable vehicle can be a solvent or dispersion medium containing, for example, water, ethanol, polyol (for example, glycerol, propylene glycol, and liquid polyethylene glycol and the like), suitable mixtures thereof, and vegetable oils. Additional examples of pharmaceutically acceptable vehicles include, but are not limited to: Water for Injection USP; aqueous vehicles such as, but not limited to, Sodium Chloride Injection, Ringers Injection, Dextrose Injection, Dextrose and Sodium Chloride Injection, and Lactated Ringers Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Prevention of the action of microorganisms in the composition can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of injectable compositions can be brought about by including in the composition an agent which delays absorption, for example, aluminum monostearate or gelatin.

In embodiments the pharmaceutical formulation comprising siRNAs molecules is a dry powder formulation to be administered by inhalation. In embodiments the pharmaceutical formulation comprises siRNAs molecules, and at least one of an excipient and a dispersion enhancer (preferably both).

In embodiments the siRNAs molecules are incorporated in a polyethylenimine (PEI) powder formulation to be administered by inhalation. Such formulations have been described for instance by Okuda et al., J. Controlled Relase, (2018), 279: 99-113 and Miwata et al., Mol Ther Nucleic Acids. (2018), 12: 698-706, which are both incorporated herein by reference.

In one particular embodiment the pharmaceutical formulation is a PEI powder formulation comprising polyethyleneimine (e.g., branched PEI, Mw 70 000, Polysciences, Inc., Warrington, USA), D-mannitol (e.g., Wako Pure Chemical Industries, Osaka, Japan) as the excipient and L-leucine (e.g., Sigma-Aldrich, St Louis, USA) as the dispersion enhancer.

Table 2 hereinafter provides particular examples of compositions for PEI powder formulations and dosages in accordance with the present invention. Reference is made to siRNA #313 for purpose of example only since similar formulation would also be applicable to other siRNAs, in accordance with the present invention.

TABLE 2
Examples of PEI powder formulations and dosages

| | Component amount* | | | | Amount of powder | | |
|---|---|---|---|---|---|---|---|
| Formulation | SIRNA | PEI | Leu | Man | 1 mg | 0.5 mg | 0.25 mg |
| Name | mg | mg | mg | mg | siRNA dose (μg) | | |
| #313PML06 | 0.3 | 0.369 | 2.5 | 46.831 | 6 | 3 | 1.5 |
| #313PML18 | 0.9 | 1.107 | 2.5 | 45.493 | 18 | 9 | 4.5 |
| #313PML30 | 1.5 | 1.845 | 2.5 | 44.155 | 30 | 15 | 7.5 |

*PEI = polyethyleneimine; Leu = L-leucine; Man = D-mannitol

The active compound, i.e. siRNA(s), may be formulated prior to administration into pharmaceutical compositions using available techniques and procedures. Formulations of the active compound may be prepared so as to provide a pharmaceutical composition in a form suitable for any route of administration such as oral administration, injections (e.g. subcutaneous, intramuscular, intrathecal, intravenous, intra-nasal), sublingual, buccal, rectal, vaginal, ocular, otic, nasal, inhalation (e.g. intratracheal, pulmonary), nebulization, cutaneous, and transdermal administration. In preferred embodiments the route of administration is inhalation and/or nebulization.

The formulation may, where appropriate, be conveniently presented in discrete dosage units and may be prepared by any of the methods well-known in the art of pharmaceutical formulation. All methods include the step of bringing together the active pharmaceutical ingredient(s) with liquid carriers or finely divided solid carriers or both as the need dictates. When appropriate, the above-described formulations may be adapted so as to provide sustained release of the active pharmaceutical ingredient. Sustained release formulations well-known to the art include the use of a bolus injection, continuous infusion, biocompatible polymers or liposomes. In preferred embodiments, the compositions according to the invention are formulated for inhalation and/or nebulization.

The methods of treatments and compositions of the present invention may also be used in combination with antiviral therapies (e.g. antivirals, anti-inflammatories, etc.) and/or vaccines, especially already approved therapies. Examples of currently approved antiviral agents, or antiviral compounds being tested in human subjects for treatment of viral infection(s) include, but are not limited to, hydroxychloroquine, ribavirin (Virazole®), lopinavir, ritonavir, remdesivir (Veklury®), favipiravir, colchicine and ivermectine, etc. For instance, Example 3 hereinafter provides evidence of an additive and possibly synergistic inhibition of virus reduction when combining siRNAs in accordance with the present invention with remdesivir (Veklury®).

Accordingly, because of sequence-specificity, the siRNA molecule should permit to minimize or avoid adverse effects to the host cells, thereby permitting a potential combination therapy.

Although the essence of the present invention aims at omitting drug delivery agents for administration of the antiviral siRNAs, it may still be possible to use the siRNA molecules of the invention with any suitable drug delivery system (DDS) reagent such as those DDS using lipid nanoparticle (LNP) and liposomes.

Those skilled in the art will recognize, or be able to ascertain, using no more than routine experimentation, numerous equivalents to the specific procedures, embodiments, claims, and examples described herein. Such equivalents are considered to be within the scope of this invention, and covered by the claims appended hereto. The invention is further illustrated by the following examples, which should not be construed as further or specifically limiting.

### Example 1: Designing and Testing Antiviral siRNA Molecules Specific to SARS-CoV-2

Because of their structure, siRNA molecules are not being incorporated easily into mammalian cells, particularly due to the paucity of electric positive charge and the lipophilic features in the molecules.

The present inventors have overcome these structural problems by synthesizing RNA molecules comprising chemically modified amino-alkyl-nucleotides (AA-Nt). Such chemically modified RNAs have been incorporated successfully into cells without the need of a drug delivery system (DDS) reagent such as lipofectamine and they have shown anticancer efficacy on intraperitonealy dispersed ovary cancers (Japanese Patent application No. 2019-148061 filed on Aug. 8^{th} 2019).

The inventors have applied this innovative technology to address the SARS-CoV-2 world pandemic. Three (3) siRNA nucleotide sequences were primarily designed for specificity to SARS-CoV-2 while avoiding “Off-target” human sequences in order to avoid any adverse effect to human cells. The determination of the sequence of the three (3) siRNA nucleotide was based on the Ui-Tei rule which was proven to deliver sequence having no adverse effect to human cells (Ui-Tei K, Naito Y, Takahashi F, Haraguchi T, Ohki-Hamazaki H, Juni A, Ueda R, Saigo K. (2004) Guidelines for the selection of highly effective siRNA sequences for mammalian and chick RNA interference. Nucleic Acids Res. 32, 936-948; Ui-Tei K, Naito Y, Nishi K, Juni A, Saigo K. (2008) Thermodynamic stability and Watson-Crick base pairing in the seed duplex are major determinants of the efficiency of the siRNA-based off-target effect. Nucleic Acids Res. 36, 7100-7109).

Particularly, the nucleotide sequence of the three siRNAs was selected from regions of the SARS-CoV-2 RNA genome encoding critical regions of the virus and they were derived mostly from the RNA-dependent RNA polymerase region (ORF-1 a and ORF-1 b; GenBank accession number NC 045512) which are considered to be essential for the virus transcription as well as the genome RNA replication. These genomic regions are also so important for RNA viruses because they are the template of mRNA, they are rarely mutated, and thus, siRNAs made with these sequences may also work for any new related coronavirus which may emerge in the future.

The three siRNA molecules that were synthesized contained: (i) a plurality of 2′-O-Methylnucleoside in both strands (i.e. P and G strands), two or three 2′-fluorinatednucleoside in the guide strand (G strand) and one 4′-aminoethyl-2′-fluorouridine in the P strand. The type, position and the number of modifications in the siRNA molecules were optimised based on the Koizumi-Kano Rule published by Koizumi et al. (2018), Synthesis of 4′-aminoalkyl-2′-O-methyl modified RNA and their biological properties, Bioorganic & Medicinal Chemistry 26,3521-3534; and by Kano et al. (2018), Synthesis and properties of 4′-C-aminoalkyl-2′-fluoro-modified RNA oligomers, Bioorganic & Medicinal Chemistry 26,4574-4582. The three optimised siRNA molecules were identified as #307§, #308§ and #309§ (see hereinbefore).

A spermine molecule was next coupled to the 5′ terminus of each of the three siRNA molecules to obtain Spermine-conjugated-aminoalkyl-uridine-built-in-anti-SARS-CoV-2-siRNAs (SP-AA-COVID-siRNA). The spermine was added in accordance to the method described in international patent application PCT/JP2019/148061 and scientific publications (Marc Nothisen, Jeremy Bagilet, Jean-Paul Behr, Jean-Serge Remy, and Mitsuharu Kotera, Mol. Pharmaceutics 2016, 13, 2718-2728; and Marc Nothisen, Mitsuharu Kotera, Emilie Voirin, Jean-Serge Remy, and Jean-Paul Behr, J. AM. CHEM. SOC. 2009, 131, 17730-17731). Briefly, these siRNAs were synthesized using the phosphoramidite method using spermine phosphoramidite on a DNA/RNA synthesizer. These resulting siRNAs were identified as siRNAs #307, #308 and #309 (FIG. 2). The siRNAS #307, #308 and #309 were next tested for their antiviral activity against SARS-CoV-2 (c.f. Example 2).

### Example 2: In Vitro Inhibition of SARS-CoV-2 Replication

The siRNA molecules designated as #307, #308, #309 were tested at different concentrations from 0-200-500-1000-1500 nM for their antiviral activity against SARS-CoV-2 in Vero-infected cells.

Briefly, a 96-multiwell plate of Vero cells was prepared 24 hours before the experiment. The siRNAs were incubated for 5 hours with the cells to prime them prior to infection. At the end of the incubation, the supernatant was taken, the Vero cells were infected and the plates incubated for 1 h at 37° C. The viral concentration corresponded to 0.05 MOI. There was 16,500 cells per well, and each well was infected with 825 pfu. Subsequently the inoculum was eliminated, washed with MEM and medium containing the respective concentrations of siRNA was added back to the wells containing the infected cells.

A background control was obtained by infecting two wells with the viruses, incubating for 1 h at 37° C., removing the virus, washing, and adding the medium of infection which was immediately collected and frozen. This was done to verify that the PCR did not show positivity not due to viral replication but only to contamination.

After about 3 days, an aliquot of supernatant was withdrawn and inactivated at 56° C. for 30 minutes. Samples were then subjected to quantitative rt-PCR amplification to measure the viral load in the cells.

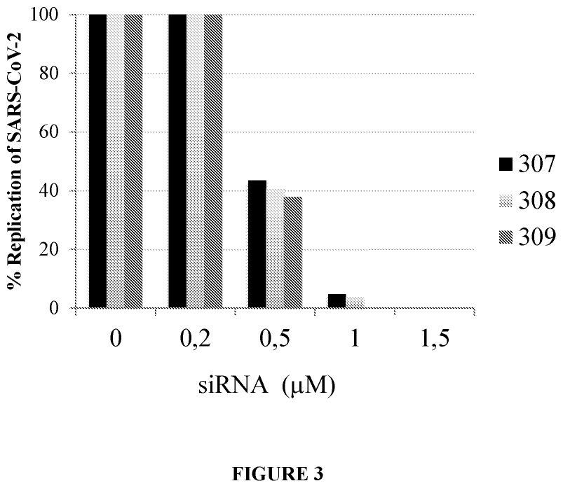

As shown in FIG. 3, the three siRNA molecules designated as #307, #308, #309 were all highly effective in inhibiting replication of SARS-CoV-2, with the strongest effects at 0.5 μM, 1 μM and 1.5 μM. siRNAS #307, #308 and #309 showed complete inhibition of SARS-CoV-2 replication in infected cells whereas controls unmodified siRNAs were not able to inhibit viral replication.

### Example 3: Antiviral Activity of siRNAS with or without Remdesivir

siRNAS #307, #308 and #309 were tested for antiviral activity alone or in combination with Remdesivir (REM) (VEKLURY®). The antiviral activity of siRNA #309 was also assed using a drug susceptibility assay.

Methods:

Compound Preparation: siRNAS #307, #308 and #309 were resuspended in a minimal volume of 2.5×PBS and diluted to 1×PBS to a stock working concentration of 250 μM. Further dilutions to experimental concentrations were made with DMEM+2% FBS.

Cells: VERO E6 cells were plated into 96-well flat bottom plates at a density 15,000 cells per well. Cells are maintained in DMEM/10% FBS/Pen/Strep up until time of infection or drug treatment. Post infection cells are maintained in DMEM/2% FBS.

Virus: SARS-CoV-2-WA1 2020 at a titer of 10\hat{ }5.8 IU/ml was used to infect VERO E6 cells. Infection was conducted in serum-free DMEM for 60 minutes with agitation every 15 minutes. Post infection virus was removed, and fresh media was replaced on the cells with or without study compounds.

Monitoring virus replication with compounds: Virus replication in the presence of compounds was monitored at 3- and 6-days post-infection. Viral infection of cells was scored by quantification of viral cytopathic effects (vCPE). At days 3 and 6 viral supernatants were harvested and viral RNA was isolated using Qiagen QIAAMP™ Viral RNA mini kits. Viral RNA was subjected to QPCR utilizing primers specific to SARS-CoV-2 spike protein.

Monitoring cell viability with compounds in the absence of virus: Cell viability was measured at day 3 and 6 utilizing Promega CellTiter-Glo™ 2.0.

Experimental Outline:

Day 1: Cells are plated into 96-well flat bottom plated at a density of 15,000 cells per well in DMEM/10% FBS/Pen/Strep.

Day 2: VERO E6 cells were pretreated with 309 compounds for 5 hours prior to infection. Compound-containing media was removed, and infection was conducted in the absence of drug for 1-hour. Virus-containing media was removed from cells and replaced with DMEM/2% FBS with drug

Day 3/6: CPE scoring was completed to quantify infection. Supernatants were harvested for QPCR analysis of SARS-Cov-2 N₁ protein. Remaining supernatants and cells were treated with CellTiter Glo™ to determine toxicity.

Results:

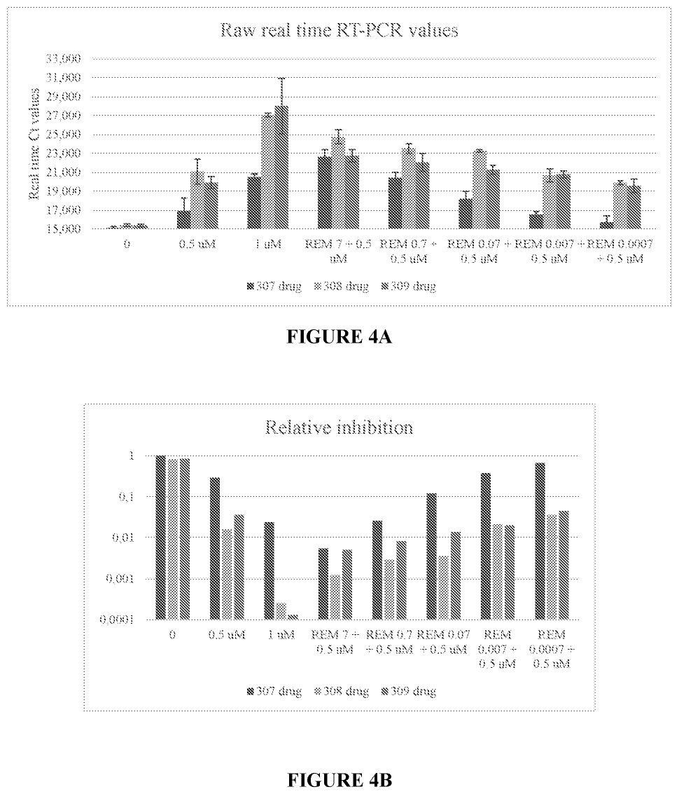

FIGS. 4A and 4B show the results of antiviral activity of siRNAs #307, #308, and #309 at a concentration of 0.5 and 1 μM with or without Remdesivir (REM). The Ct values for the qRT-PCR as plotted in FIG. 4A and the relative inhibition derived from these Ct values is shown FIG. 4B. siRNA #307 had the least antiviral potency with approximately 3.5-fold inhibition of the SARS-CoV-2 Wuhan virus at 0.5 μM and 41-fold inhibition with 1 μM. In contrast, siRNA #308 and #309 were highly active inhibitors with nearly a 30-to 60-fold decrease with 0.5 μM and at a least a 4000-fold decrease with 1 μM.

In combination with Remdesivir at 0.07 to 7 μM with 0.5 μM for #307, #308, and #309 there was at least additive if not synergistic inhibition by 0.5 μM of #307, #308, and #309 siRNAs. Concentrations of 0.007 and 0.0007 μM REM alone did not inhibit SARS-CoV-2 Wuhan. The EC50 of REM is approximately 0.8 μM in VERO-E6 cells. Concentrations of 0.7 μM REM alone results in a 1.5-fold reduction in SARS-CoV-2 replication and 0.5 μM 307, 308, and 309 siRNA alone, a 3.5-fold, 60-fold and 30-fold reduction, respectively. By combining 0.7 μM REM with 0.5 μM 307, 308, and 309, we observed above an additive and possibly synergistic inhibition with a virus reduction of 40-, 340-, and 122-fold, respectively. This study confirms the siRNAs of the present invention may advantageously be combined with other antiviral agents and antiviral therapies.

A second study was carried out to assess the antiviral activity of siRNA #309 using a drug susceptibility assay. siRNA #309 was diluted 5-fold from 50 μM to 0.64 nM and added VERO-E6 cells with or without SARS-CoV-2 Wuhan virus. Virus production was monitored at 3 and 6 days post infection using qRT-PCR and cell viability using Promega CellTiter Glo 2.0 assays.

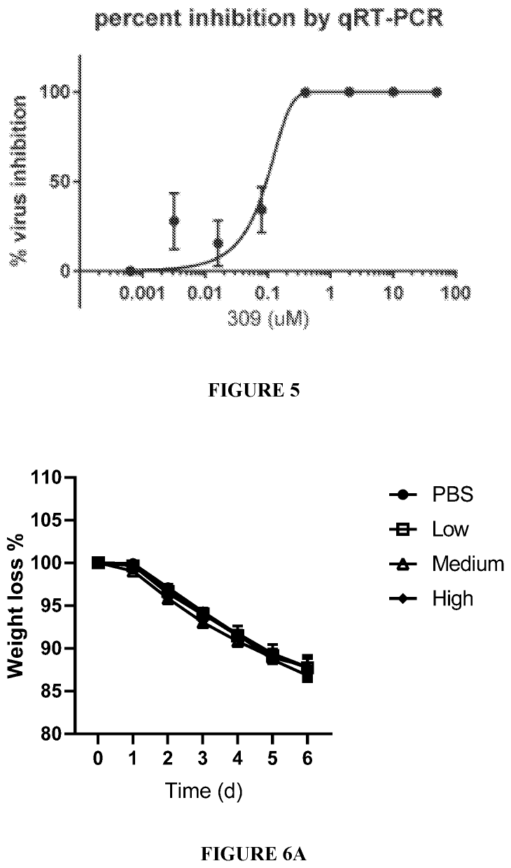

As shown in FIG. 5, siRNA #309 from 50 to 0.4 μM completely inhibited SARS-CoV-2 replication with an estimated EC50 value of 0.2 μM, which was consistent with the inhibition of 0.5 and 1 μM in the earlier study.

### Example 4: In Vivo Efficacy of siRNA #309

### SUMMARY

siRNA #309 was tested in a Syrian hamster animal model. Hamsters were treated in three cohorts, using different doses of Compound #309. Hamsters were infected with SARS-CoV-2 for five days, after which hamsters were euthanized and lung tissue removed for PCR assay and plaque assay. PCR assay showed 160-fold decrease (99.375%) in viral titer in hamsters treated with the “high” dose of Compound 309 and 4-fold decrease (80%) in viral titer in hamsters treated with the “medium” dose of Compound #309.

Experimental Method

Consistent with other infectious models for Syrian hamsters, the hamsters were treated with three different dosages 6 hours before and 12 hours after infection, followed by 5 days of incubation with SARS-CoV-2, including a third treatment 36 hours after infection. The dosing protocol is summarised in Table 3:

TABLE 3
Dosing protocol for testing siRNA #309 in Syrian hamsters

| | | 6 hr before | 12 hr post- | 36 hr post- | Total compound | |
|---|---|---|---|---|---|---|
| | Number | infection | infection | infection | (dose × 6 | |
| | of | (administration | (administration | (administration | hamsters × 3 | |
| GROUP | Hamsters | #1) | #2) | #3) | time points) | |
| Control | 6 | 100 ul PBS | 100 ul PBS | 100 ul PBS | 0 | mg |
| (no therapy) | | | | | | |
| Low | 6 | 0.045 mg in | 0.045 mg in | 0.045 mg in | 0.81 | mg |
| | | 100 ul PBS | 100 ul PBS | 100 ul PBS | | |
| Middle | 6 | 0.225 mg in | 0.225 mg in | 0.225 mg in | 4.05 | mg |
| | | 100 ul PBS | 100 ul PBS | 100 ul PBS | | |
| High | 6 | 0.45 mg in | 0.45 mg in | 0.45 mg in | 8.1 | mg |
| | | 100 ul PBS | 100 ul PBS | 100 ul PBS | | |
| | | | | TOTAL | 12.96 | mg |

Hamsters received compound via intranasal administration (in 100 μI PBS) at the three indicated administration time points. Infection with SARS-Cov-2 (1×10⁶ PFU per hamster) occurred 6 hrs. following the first dose.

Five days following infection, hamsters were euthanized and lung tissue removed for histological examination, PCR assay (live or dead virus), and plaque assay (specifically live virus). Lungs were homogenized, then centrifuged at 1500 RPM for 10 minutes to clarify supernatants. Supernatants were removed and viral titers quantified by limiting dilution on VeroE6 cells in 24 well plates.

Results

Eight-week-old Syrian hamsters were treated and infected as described above. Weights were taken daily to assess morbidity and animals were monitored for physical signs of illness. On day 5 post-infection, lungs were harvested and processed for viral titers.

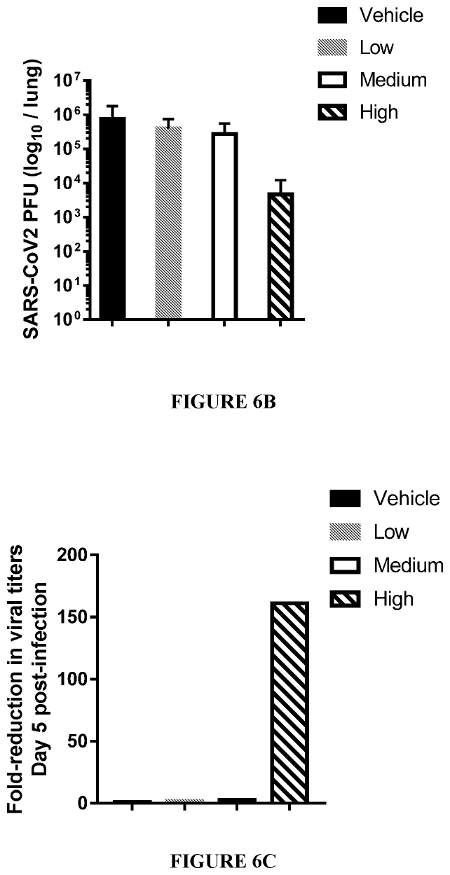

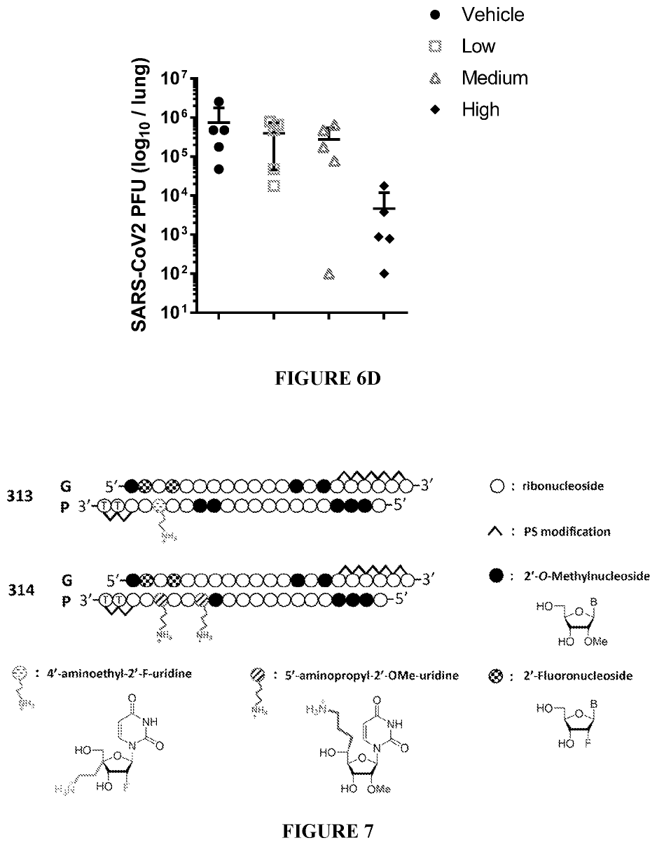

Results are shown in FIGS. 6A-6D. As shown in the figures, in the medium and high dose groups of animals treated with siRNA #309, the viral content of the hamsters' lung tissue was reduced by 80% to 99.375%. In two out of twelve animals in those groups, the virus was eliminated entirely. In the “high” dosage cohort, the average reduction in viral titer was 163-fold by PCR analysis (over 2 logs by plaque assay), with one animal showing complete viral elimination. This effect on a virus at an initial trial, especially prior to either the standardization of the dosage or even a refinement of the delivery protocol, is rare and a proof of a very strongest antiviral activity.

### Example 5: Clinical Development of siRNA Candidates Specific to SARS-CoV-2

It is within the skills of those in the art to screen, select and develop potential antiviral siRNA candidates for treatment of antiviral diseases in humans, in accordance with the present invention. For instance, for testing and development of an antiviral agent specific to SARS-CoV-2, the following approach could be taken.

Stage I: Initial screening of antiviral activity and cell cytotoxicity of the modified siRNA candidates are performed in Vero cells using GFP labeled SARS-CoV-2 virus (minus virus for toxicity analyses). Virus replication is to be monitored over 7 days in cells preincubated for one day with 5-fold dilutions of the siRNA. Results are reported as EC50 and CCID50 values. The best siRNA candidates are next tested in a follow-up drug inhibition experiment using the lung epithelial cell line, Calu-3.

Stage II. Animal testing are performed with the best 2 or 3 candidates using the Syrian hamster model. The hamsters are infected with SARS-CoV-2-luc (i.e., comprising a carrying luciferase reporter useful to monitor the virus in real time.). Infection and drug protection is monitored by a combination of viral-encoded fluorescence in hamsters and tracking replication in various tissues by non-invasive IVIS-CT. Mucosal and blood samples are also obtained to measure viral load by RT-PCR.

Stage III. It is determined if nebulized siRNA into the hamster lungs can provide protection against an aerosolized SARS-CoV-2 luc challenge to the lungs. After administration of the nebulized siRNAs, the hamsters are placed in an aerosolization chamber for SARS-CoV-2 challenge using physiological dosing.

Stage II and/or Stage III animals testing can also be done in alternative acceptable animal models, including but not limited to the ACE2 transgenic (ACE2 Tg) mice. Various delivery methods can also be tested, including but not limited to, intratracheal administration, pulmonary administration, powder formulation for inhalation, spray-dry methods, etc.

Stage IV. Candidates siRNAs showing animal in vivo efficacy are next tested in humans by way of clinical trials, according to standard protocols approved by the regulatory authorities (e.g. FDA).

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a molecule” or “a siRNA” includes one or more of such molecules or siRNAs and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible.

Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. An antiviral silencing RNA molecule (siRNA), wherein said antiviral siRNA molecule comprises a nucleotide sequence that targets a virus, wherein said antiviral siRNA molecule is a double-stranded RNA molecule comprising a P strand and a G strand, wherein said antiviral siRNA molecule comprises 19 to 25 base pairs, wherein said antiviral siRNA can penetrate mammalian infected cells without assistance of a drug delivery system (DDS) reagent, and wherein said antiviral siRNA can inhibit and/or silence expression and/or translation of genes from said virus.

2-5. (canceled)

6. The antiviral siRNA of claim 1, wherein said antiviral siRNA molecule further comprises 2, 3, 4, or 5 TT nucleotides overhangs at its 5′- or at its 3′-end.

7. The antiviral siRNA of claim 1, wherein said antiviral siRNA comprises a nucleotide sequence selected from the group consisting of siRNAs #301, #302 and #303:

| | SEQ | |
|---|---|---|
| SIRNA | ID | |
| # | NO: | SEQUENCES |
| 301 | 1 | G: 5′-AAU UAU UAA CCA CAU AAG CCA-3′ |
| | 2 | P: 3′-AA UUA AUA AUU GGU GUA UUC G-5′ |
| 302 | 3 | G: 5′-UAA UUC UAA GCA UGU UAG GCA-3′ |
| | 4 | P: 3′-GU AUU AAG AUU CGU ACA AUC C-5′ |
| 303 | 5 | G: 5′-UCA UAA ACG GAU UAU AGA CGU-3′ |
| | 6 | P: 3′-UU AGU AUU UGC CUA AUA UCU G-5′ |

8. The antiviral siRNA of claim 1, wherein said antiviral siRNA comprises at least one chemically modified nucleotide selected from the group consisting of 2′-O-Methylnucleoside, 2′-Fluoronucleoside, aminoalkyl-nucleotide, aminoethyl-nucleotide, 5′-aminopropyl-2′-OMe-nucleoside and combinations thereof.

9-10. (canceled)

11. The antiviral siRNA of claim 8, wherein said at least one chemically modified nucleotide is present in the P strand of the antiviral siRNA.

12. The antiviral siRNA of claim 1, wherein said antiviral siRNA comprises a nucleotide sequence selected from the group consisting of siRNAs #304, #305 and #306:

| SIRNA | SEQ ID | |
|---|---|---|
| # | NO: | SEQUENCES* |
| 304 | 7 | G: 5′-aAU UAU UAA CCA cAu A•A•G•C•C•A-3′ |
| | 8 | P: 3′-T•T•uuA AUA AuU GGU GUA uuc G-5′ |
| 305 | 9 | G: 5′-uAA UUC UAA GCA uGu U•A•G•G•C•A-3′ |
| | 10 | P: 3′-T•T•uu AAG AuU CGU ACA Auc C-5′ |
| 306 | 11 | G: 5′-uCA UAA ACG GAU uAu A•G•A•C•G•U-3′ |
| | 12 | P: 3′-T•T•AGu AUu uGC CUA AUA ucu G-5′ |

wherein
a nucleotide in lowercase represents a 2′-O-Methylnucleoside;
a nucleotide in bold represents a 2′-Fluoronucleoside; and
a dot (⋅) represents a phosphorothioate (PS) bond.

13. The antiviral siRNA of claim 1, wherein said antiviral siRNA comprises a nucleotide sequence selected from the group consisting of siRNAs #307§, #308§ and #309§ as follows:

| SIRNA | SEQ | |
|---|---|---|
| # | ID NO: | SEQUENCES* |
| 307§ | 13 | G: 5′-aAU UAU UAA CCA cAu A•A•G•C•C•A-3′ |
| | 14 | P: 3′-T•T•uuA AUA AuU GGU GUA uuc G-5′ |
| 308§ | 15 | G: 5′-uAA UUC UAA GCA uGu U•A•G•G•C•A-3′ |
| | 16 | P: 3′-T•T•Auu AAG AuU CGU ACA Auc C-5′ |
| 309§ | 17 | G: 5′-uCA UAA ACG GAU uAu A•G•A•C•G•U-3′ |
| (313) | 18 | P: 3′-T•T•AGu AUu uGC CUA AUA ucu G-5′ |

wherein
a nucleotide in lowercase represents a 2′-O-Methylnucleoside;
a nucleotide in bold represents a 2′-Fluoronucleoside;
an underlined uridine represents 4′-aminoethyluridine-2′-F; and
a dot (⋅) represents a phosphorothioate (PS) bond.

14. The antiviral siRNA of claim 1, wherein said antiviral siRNA comprises a nucleotide sequence consisting of siRNA #314 as follows:

| SIRNA | SEQ | |
|---|---|---|
| # | ID NO: | SEQUENCE* |
| 314 | 25 | G: 5′-uCA UAA ACG GAU uAu A•G•A•C•G•U-3′ |
| | 26 | P: 3′-T•T•AG [symbol] AU [symbol] uGC CUA AUA ucu G-5′ |

wherein
a nucleotide in lowercase represents a 2′-O-Methylnucleoside;
a nucleotide in bold represents a 2′-Fluoronucleoside;
a uridine in small cap italic represents a 5′-aminopropyl-2′-OMe-uridine; and
a dot (⋅) represents a phosphorothioate (PS) bond.

15-17. (canceled)

18. The antiviral siRNA of claim 1, wherein said antiviral siRNA comprises any one of SEQ ID NOs: 1 to 26.

19. An antiviral siRNA selected from the group consisting of siRNAs #313 and #314:

| SIRNA | SEQ ID | |
|---|---|---|
| # | NO: | SEQUENCE |
| 313 | 17 | G 5′-uCA UAA ACG GAU uAu A•G•A•C•G•U -3′ |
| (309§) | 18 | P: 3′-T•T•AGu AUu uGC CUA AUA ucu G -5′ |
| 314 | 25 | G: 5′-uCA UAA ACG GAU uAu A•G•A•C•G•U-3′ |
| | 26 | P: 3′-T•T•AG [symbol] AU [symbol] uGC CUA AUA ucu G-5′ |

wherein
a nucleotide in lowercase represents a 2′-O-Methylnucleoside;
a nucleotide in bold represents a 2′-Fluoronucleoside;
an underlined uridine represents 4′-aminoethyluridine-2′-F; and
a uridine in small cap italic represents a 5′-aminopropyl-2′-OMe-uridine; and
a dot (⋅) represents a phosphorothioate (PS) bond.

20. (canceled)

21. An isolated nucleic acid molecule, comprising any one of SEQ ID NOs: 1 to 26.

22-35. (canceled)

36. A pharmaceutical composition comprising an antiviral siRNA as defined in claim 1, and at least one pharmaceutically acceptable carrier, diluent, vehicle, excipient and/or dispersion enhancer.

37. The pharmaceutical composition according to claim 36, wherein said pharmaceutical composition is formulated as a dry powder formulation for administration by inhalation.

38. The pharmaceutical composition according to claim 36, wherein said pharmaceutical composition comprises siRNAs molecules, polyethyleneimine, an excipient and a dispersion enhancer.

39. The pharmaceutical composition according to claim 38, wherein the excipient comprises D-mannitol and the dispersion enhancer comprises L-leucine.

40-42. (canceled)

43. A method for treating a viral infection, comprising administering to a subject in need a therapeutically effective amount of one or more of an antiviral siRNA as defined in claim 1.

44. The method of claim 43, wherein administering said one or more antiviral siRNA comprises a route of administration selected from the group consisting of oral, injection, sublingual, buccal, rectal, vaginal, ocular, otic, nasal, inhalation, nebulization, cutaneous, and transdermal administration.

45. The method of claim 44, wherein the route of administration is inhalation or nebulization.

46. The method of claim 43, wherein said antiviral siRNA, said isolated nucleic acid molecule or said pharmaceutical composition, is administered in combination with a drug or a vaccine.

47. The method of claim 46, wherein the drug is selected from the group consisting of hydroxychloroquine, ribavirin, lopinavir, ritonavir, remdesivir, favipiravir, colchicine, and ivermectine.

48-51. (canceled)

52. The method of claim 43, wherein the viral infection is an infection by a corona virus.

53. The method of claim 52, wherein the corona virus is selected from the group consisting of SARS-CoV, SARS-CoV-1, MERS-CoV, and SARS-CoV-2.

54. The method of claim 53, wherein said virus is SARS-CoV-2 and wherein said antiviral siRNA comprises a nucleotide sequence that is complementary with the RNA-dependent RNA polymerase region of SARS-CoV-2.
